(19) United States
(12) Patent Application Publication
(10) Pub. No.: US 2020/0275989 A1
(43) Pub. Date: Sep. 3, 2020

(54) FLEXIBLE HOLDING DEVICE

(71) Applicant: OTTO-VON-GUERICKE-UNIVERSITÄT MAGDEBURG, Magdeburg (DE)

(72) Inventors: Juan Sebastian SANCHEZ LOPEZ, Magdeburg (DE); Robert ODENBACH, Magdeburg (DE); Michael FRIEBE, Recklinghausen (DE)

(73) Assignee: Otto-von-Guericke-Universität Magdeburg, Magdeburg (DE)

(21) Appl. No.: 16/645,514

(22) PCT Filed: Sep. 10, 2018
(86) PCT No.: PCT/EP2018/074277

§ 371 Date: Mar. 9, 2020

(30) Foreign Application Priority Data
Sep. 8, 2017 (DE) 10 2017 120 771.6

(51) Int. Cl. A61B 90/50

(52) U.S. Cl. CPC A61B 90/50; A61B 2090/374

(57) ABSTRACT

The invention relates to a holding device comprising at least one locking unit (2) having at least two sockets (3), wherein the sockets (3) are designed to make it possible for joint heads (5) received in the sockets (3) to each pivot about a pivot axis, as well as to a locking unit, to a tool mount and to an instrument holder for use in magnetic resonance imaging (MRI).

The invention relates to a flexible holding device, in particular a flexible holding device for preferred use in magnetic resonance imaging (MRI), as well as to a locking unit, to a tool mount and to an instrument holder for use in magnetic resonance imaging (MRI).

The current possibilities of MRI have not yet been exhausted, especially in interventional applications. This is mainly due to the fact that not enough medical devices are available that can be used in MRI. In this context, many patients could benefit from an operation conducted using MRI, for example when taking tissue samples. Nowadays, such biopsies are often carried out with the assistance of ultrasound (US) or computer tomography (CT), although these methods are considerably inferior to the options afforded by MRI. This relates in particular to the detectability of different tissues, which can be significantly increased by means of MRI, and thus to the precision of taking samples. Providing more MRI-compatible instruments would result in a significant improvement in treatment options.

In the prior art, various instruments and holding devices are known for medical use and specifically for use in the MRI sector. A drawback of these instruments and thus the prior art is that the known holding devices, in particular in the MRI sector, are highly complex in structure. This means that the appliances cannot be operated without prior training. In addition, the complicated handling increases the length of time the patient has to spend in the MRI, which is undesired and disadvantageous both for the patient and the hospital. Not least, known holding devices are often very expensive, because of the technology used. Known appliances thus have a large number of options that are often neither used nor desired. These additional options result in the above-mentioned drawbacks, such as difficult, counter-intuitive operation, reduced patient safety, and high purchase and maintenance costs.

The problem addressed by the invention is therefore to provide a holding device, a locking unit, a tool mount, and an instrument holder which are easy to handle and at the same time provide comprehensive options for positioning.

Another problem addressed by the invention is therefore to provide a holding device, a locking unit, a tool mount, and an instrument holder which are particularly suitable for use in magnetic resonance imaging (MRI). In addition, they can be used with other diagnostic systems and as a device for assistance during operations.

This problem is solved by an invention having the features of claims 1, 15, 19 and 22. Advantageous embodiments are found in the dependent claims. It should be noted that the features that are set out individually in the claims can also be combined with one another in any technologically viable manner, and thus demonstrate further embodiments of the invention.

A holding device according to the invention comprises at least one locking unit comprising at least two sockets. The holding device may further comprise at least one locking unit comprising a socket. The sockets are designed to make it possible for joint heads received in the sockets to each pivot about at least one pivot axis, and preferably precisely one pivot axis. Embodiments comprising a plurality of pivot axes are also conceivable, however.

An advantageous configuration provides that the sockets are designed to be rotatable relative to one another such that the orientation of the pivot axes of the sockets relative to one another can be changed. This can be achieved for example by the locking unit being divided between the two sockets and the two parts of the locking unit being designed to be rotatable relative to one another. For this purpose, a cuff which comprises two circumferential notches on the side facing the locking unit can be placed around the two-part locking unit in the region of the division, for example. The two parts of the locking unit engage in this notch in a form-fitting manner with outwardly directed projections, which notch thus serves as a guide rail for the rotational movement of the two parts of the locking unit. Other options for configuring rotation of the sockets relative to one another according to the invention are known to a person skilled in the art.

In a preferred embodiment, the holding device comprises at least one connector element having at least two joint heads, wherein at least one joint head of the connector element is received in a socket of the locking unit.

In a particular configuration, the holding device preferably comprises at least two locking units and at least one connector element, wherein the joint heads of the connector element are each received in a socket of a locking unit.

The socket and joint heads may for example be designed as ball-and-socket joints, but other types of joint are also conceivable, and a person skilled in the art would select a suitable joint construction here.

The invention further relates to a locking unit, as is described in greater detail above and in the following. The locking unit comprises at least two sockets, wherein the sockets are designed to make it possible for joint heads received in the sockets to each pivot about at least one pivot axis, and preferably precisely one pivot axis. Furthermore, the locking unit comprises at least two locking elements, wherein the locking elements lock joint heads received in the sockets in a locking position and allow joint heads received in the sockets to move freely about the pivot axes in an open position. Furthermore, locking units comprising one socket are also conceivable.

An advantageous configuration of the locking unit provides that the sockets are designed to be rotatable relative to one another in an open position such that the orientation of the pivot axes of the sockets relative to one another can be changed. The locking elements lock the sockets relative to one another in the locking position, such that the position of the pivot axes can be fixed in the locking position.

In order to make it possible to adjust the joint heads by up to 90° relative to the longitudinal axis of the locking unit, the sockets may comprise a cut-out at the ends of the locking unit which is suitable for receiving the shaft of the joint heads.

A particular embodiment of the locking unit provides that, in the locking position, the locking elements are pressed against joint heads received in the sockets by eccentrically arranged eccentric elements of a locking lever. By means of this pressure, both the joint heads are fixed in the sockets and the position of the pivot axes relative to one another is also fixed.

The substantially rod-shaped locking lever is preferably arranged perpendicularly to the longitudinal axis of the locking unit at a point between the two sockets of the locking unit. The two ends of the locking lever are rotatably mounted in a tubular casing of the locking unit, at least the first end also penetrating through the casing towards the outside. This first end comprises a rotary lever or at least an attachment point for a rotary lever in the region of the outside of the casing. The portion of the locking lever inside the cavity in the casing comprises at least two eccentric projections, which are opposite one another based on the axis of the locking element. If the locking lever is then rotated by 90° by means of the rotary lever, the eccentric projections can be moved from a position along the longitudinal axis of the locking unit, referred to in the following as the locking position, into a position perpendicular to the longitudinal axis of the locking unit, referred to in the following as the open position.

A movably mounted locking element, the external diameter of which preferably substantially corresponds to the internal diameter of the tubular casing, is positioned on either side of the locking lever within the tubular casing. The length of the locking element is coordinated with the eccentric projections of the locking lever such that the locking elements do not lock the joint heads and pivot axes in the open position and the locking elements lock the joint heads and pivot axes in the locking position of the locking lever. The locking is carried out by the locking elements being pushed against the joint heads by the eccentric projections of the locking element, as a result of which said joint heads are in turn pushed against the sockets of the locking unit and the projections of the two parts of the locking unit are pushed into the notch. Accordingly, by adjusting the locking lever simultaneously using a handle, both locking elements are moved and are locked or released using a handle. In particular, the ends of the locking elements that extend as far as the joint heads may be coated with a material that additionally prevents the joint heads from slipping, for example an elastomer. Alternatively, the material of the locking elements may consist of an elastomer in part. Other options for the locking are known to a person skilled in the art.

In another preferred embodiment, the locking unit is adjustable in length between the sockets.

An advantageous configuration of the locking unit provides that the locking unit comprises a casing made up of at least two casing parts, wherein at least two casing parts are designed to be adjustable relative to one another for the length adjustment of the locking unit.

A particular configuration of the locking unit provides that the at least two casing parts can be latched into one another by latching elements.

In addition, according to an advantageous configuration of the locking unit, at least one of the locking elements comprises a first and a second locking element part, wherein the first and the second locking element part are designed to be adjustable relative to one another for the length adjustment of the locking unit and can be latched into one another by latching elements.

According to a particular configuration, the latching elements of the casing parts and the locking element parts are preferably designed as form-fitting elements that are formed in part in the circumferential direction. These form-fitting elements may for example be formed as screw threads, hooks or teeth, wherein the latching elements may also be designed as teeth that are formed in the longitudinal direction in part.

According to a particularly advantageous embodiment, the latching elements are arranged on the corresponding casing parts and locking element parts such that at least one casing part and one of the locking element parts can be adjusted together relative to the other casing and locking element parts. By the coupling of the length adjustment of casing parts and locking element parts, when adjusting the length of the locking unit, locking is also made possible by means of the locking elements, since the locking element changes its length analogously to the length adjustment of the casing.

In another preferred embodiment, the holding device comprises a tool mount as described in the following. The tool mount described is preferably intended for use on a holding device according to the invention, but it is not limited thereto.

The tool mount comprises at least one hole for receiving a tool. In a closed position, at least one locking shaft engages in the hole and the tool can thus be fixed in the hole. In an open position, the locking shaft does not engage in the hole and the tool can be removed from the hole.

According to an advantageous configuration of the tool mount, the locking shaft can preferably be moved from a closed position into an open position by an unlocking mechanism that can be released by an unlocking element.

According to a particular configuration, the unlocking mechanism comprises a spring, which moves the locking shaft into the closed position by means of spring force.

In another preferred embodiment, the holding device also comprises, in addition to the tool mount, an instrument holder as described in the following. The instrument holder described is preferably intended for use with the holding device according to the invention, but it is not limited thereto.

The instrument holder according to the invention comprises a housing, in which at least one rotatable flat thread is arranged. A plurality of sliders, preferably three sliders, are arranged on the flat thread and penetrate through the housing through first openings in the housing. The first openings in the housing are preferably designed as T-shaped guide rails, which extend in the manner of spokes in a straight line from the central cut-out to the edge of the cylinder. The three guide rails are preferably arranged at an angle of 120° relative to one another. A slider runs in each of the T-shaped guide rails. Each slider comprises lateral protrusions, which are received by the T-shaped guide rails of the cover. The sliders can be moved towards one another by rotation of the flat thread in a first rotational direction and can be moved away from one another by rotation of the flat thread in a second rotational direction counter to the first rotational direction.

According to an advantageous configuration of the instrument holder, the flat thread has rotary means that make it possible to rotate the flat thread. The rotary means are preferably accessible from the outside via at least one second opening in the housing of the instrument holder.

Cut-outs may preferably be arranged centrally in the base and cover of the housing and at a corresponding point in the flat thread such that an instrument to be clamped, for example a needle, in particular a biopsy needle, can extend through this cut-out in the instrument holder.

One end of the slider, the inner end, which is within the housing, extends into the flat thread and engages with a protrusion therein. The other end of the slider, the outer end, extends out of the housing. The slider comprises gripping surfaces towards the center of the instrument holder, which surfaces can be enlarged in order to provide a larger projecting surface for holding an instrument.

The instrument holder described is preferably intended for use with the holding device according to the invention, but it is not limited thereto.

The holding device according to the invention may also comprise a fixing unit. The fixing unit comprises at least one joint head for making it possible to connect the fixing unit to a socket of a locking unit of the holding device in an articulated manner. Furthermore, the fixing unit comprises means for fixing the fixing unit to a surface underneath and thus serves to detachably fasten the entire holding device to said surface. A person skilled in the art will select the means used for fixing depending on the surface to which the fixing unit is to be detachably fastened. These means may for example be screw connections, clamped connections, suction connections, or other connections.

In another preferred embodiment, the holding device as well as the tool mount and the instrument holder are suitable for use in MRI. This is achieved by selecting suitable materials during production. A particularly preferred material here is polycaprolactam (nylon 6), but many other materials that are known to a person skilled in the art are also suitable for use in MRI.

The invention and the technical field are described in greater detail in the following with reference to the drawings. It should be noted that the drawings show a particularly preferred variant of the invention; however, the invention is not limited to the variant shown. Provided that it is technically viable, the invention in particular includes any combination of the technical features that are set out in the claims or are described in the description as being relevant to the invention.

In the drawings:

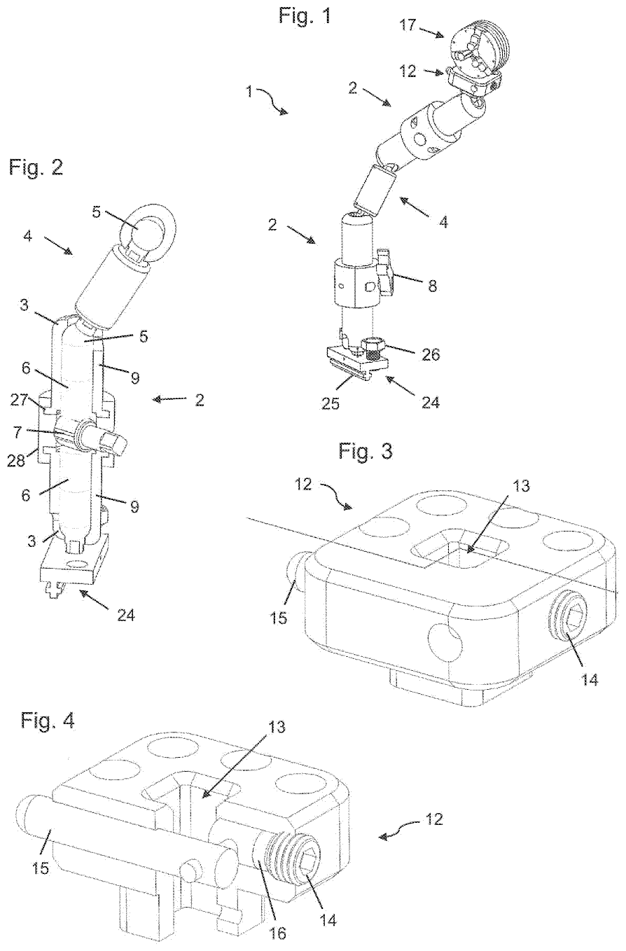

FIG. 1 is a view of a first embodiment of the holding device according to the invention.

FIG. 2 is a longitudinal section through a first embodiment of the locking element comprising a connecting element.

FIG. 3 is a view of the tool mount according to the invention.

FIG. 4 is a partial cross section through the tool mount.

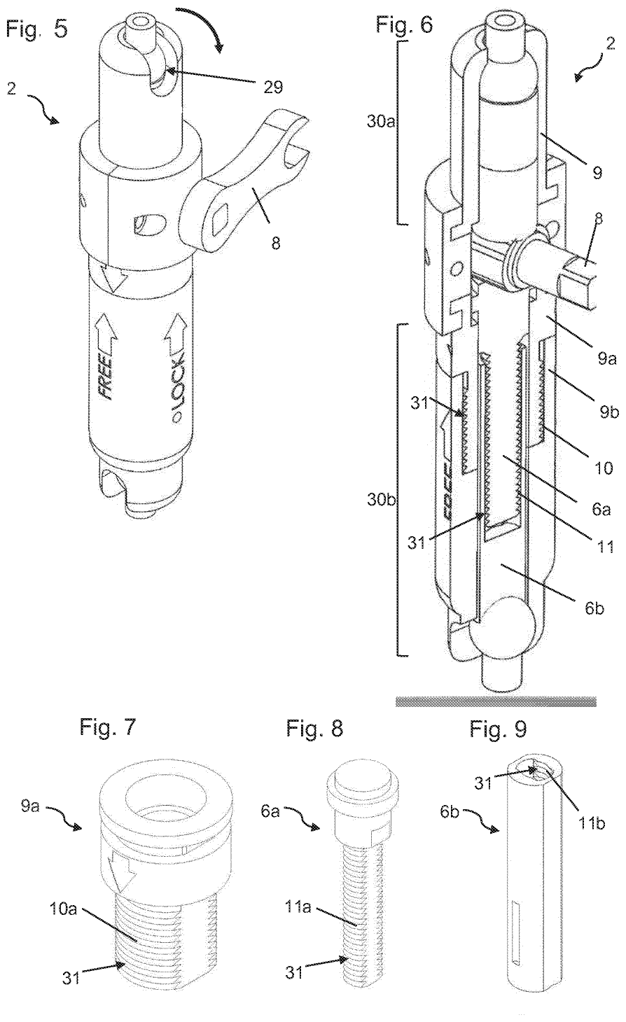

FIG. 5 is a view of a second embodiment of the locking element.

FIG. 6 is a longitudinal section through the second embodiment of the locking element.

FIG. 7 is a view of a detail of a first casing part.

FIG. 8 is a view of a detail of a first locking element part.

FIG. 9 is a view of a detail of a second locking element part.

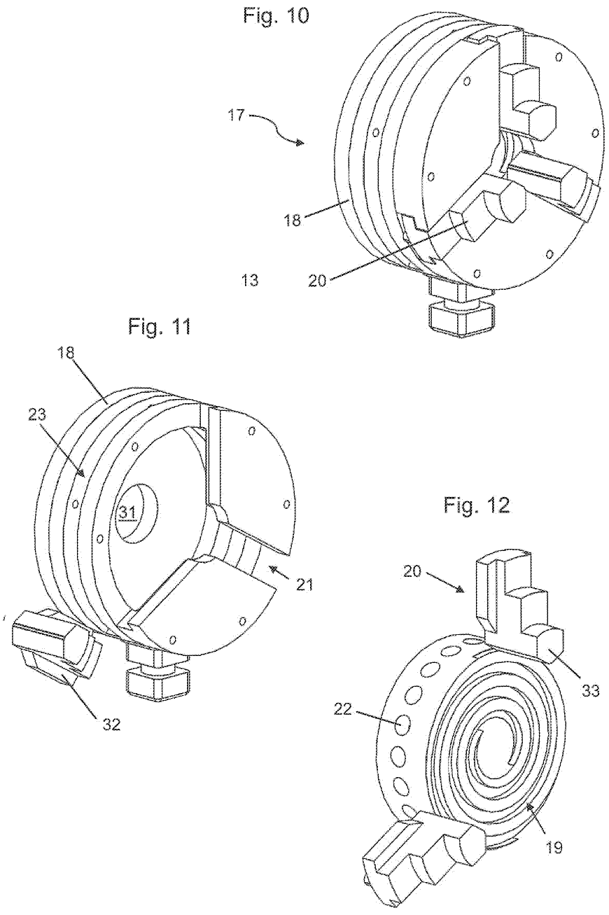

FIG. 10 is a view of a preferred embodiment of the instrument holder.

FIG. 11 is a partial section through the housing of a preferred embodiment of the instrument holder.

FIG. 12 shows the flat thread of the instrument holder together with two sliders.

FIG. 1 shows a first embodiment of the holding device 1 according to the invention, comprising a connector element 4, two locking units 2, a tool mount 12, an instrument holder 17 and a fixing unit 24. The fixing unit 24 is suitable for being fastened to a surface by means of a guide and a screw. A person skilled in the art will select suitable means depending on the surface. The fixing unit 24 also has a joint head 5 (FIG. 2), which engages in a socket 3 (FIG. 2) of one locking unit 2. The locking element 2 is connected to a second locking element 2 by a connector element 4. The connector element 4 comprises two joint heads 5 (FIG. 2), which each engage in one socket 3 (FIG. 2) of the two locking units 2. The locking units 2 are each locked by a locking lever 8, with only one being shown in FIG. 1. A tool mount 12 that has already been described and is described in greater detail in the following is received in a socket of one locking unit 2 by its own joint head. An instrument holder 17 that has already been described and is described in greater detail in the following is received in the tool mount 12 shown.

FIG. 2 is longitudinal section through a first embodiment of the locking unit 2. The substantially rod-shaped locking lever 7 is preferably arranged perpendicularly to the longitudinal axis of the locking unit 2 at a point between the two sockets 3 of the locking unit 2. The two ends of the locking lever 7 are rotatably mounted in the tubular casing 9 of the locking unit 2, with at least the first end also penetrating through the casing 9 towards the outside. This first end comprises a rotary lever 8 or at least an attachment point for a rotary lever in the region of the outside of the casing. The portion of the locking lever 8 inside the cavity in the casing comprises at least two eccentric projections 7, which are opposite one another based on the axis of the locking element. If the locking lever 8 is then rotated by 90°, the eccentric projections 7 can be moved from a locking position into an open position. A movably mounted locking element 6, the external diameter of which preferably substantially corresponds to the internal diameter of the tubular casing 9, is positioned on either side of the locking lever 8 within the tubular casing 9. The length of the locking element 6 is preferably coordinated with the eccentric projections 7 of the locking lever 8 such that the locking elements 6 do not lock the joint heads 5 and pivot axes in the open position and the locking elements 6 lock the joint heads 5 and pivot axes in the locking position of the locking lever 8. The locking is carried out by the locking elements 6 being pushed against the joint heads 5 by the eccentric projections 7 of the locking element 8, as a result of which said joint heads are in turn pushed against the sockets 3 of the locking unit 2 and the projections 27 of the two parts of the locking unit 2 are pushed into the notch 28. Accordingly, by adjusting the locking lever 8 simultaneously using a handle, both locking elements 6 are moved and are thus locked or released.

FIG. 3 shows a preferred embodiment of the tool mount 12, comprising a square hole 13, a locking shaft 14 and an unlocking element 15.

FIG. 4 is a partial section through the tool mount 12 according to FIG. 3, comprising a square hole 13, a locking shaft 14, an unlocking element 15 and a spring 16. The locking shaft 14 engages in the hole 13 with one end and thus fixes a tool inserted therein. In order to release the fixing, the locking shaft 14 is moved out of the hole 13 by means of the unlocking element 15. A spring 16 moves the locking shaft 14 into the closed position such that, without pressure on the unlocking element 15, the tool mount 12 remains in the closed position.

FIG. 5 shows another preferred embodiment of the locking unit 2 having length adjustment. The joint ball 5 (FIG. 2) can be pivoted in at least one axis 29 in the socket 3 (FIG. 2). The joint ball 5 (FIG. 2) can, however, be adjusted in a plurality of pivot axes. The axis 29 defined by the joint ball 5 (FIG. 2) and the socket 3 (FIG. 2) can be rotated for this purpose. In addition, depending on the opening width of a pocket formed in the socket 3 (FIG. 2), the joint ball 5 (FIG. 2) can be rotated relative to the socket 3 (FIG. 2) in all three spatial axes within a certain range. The ability to pivot about the axis 29 is limited by means of the lateral guides in the socket 3 (FIG. 2). By means of a wider opening in the socket 3 (FIG. 2), the joint ball 5 (FIG. 2) and the socket 3 (FIG. 2) can also pivot all the way around. This feature is also implemented in the embodiment described above in FIGS. 1 and 2.

FIG. 6 is a longitudinal section through the preferred embodiment of the locking unit 2 having length adjustment. The locking unit 2 comprises a casing made up of two casing portions 30a, 30b, with the casing portion 30a being formed in one piece and the casing portion 30b being formed in at least two pieces. The casing parts 9a, 9b of the casing portion 30b are designed to be adjustable relative to one another for the length adjustment of the locking unit 2 and can be latched into one another by first latching elements 10. In addition, at least one of the locking elements 6 of the locking unit 2 comprises a first and a second locking element part 6a, 6b, wherein the first and the second locking element part 6a, 6b are likewise designed to be adjustable relative to one another for the length adjustment of the locking unit 2 and can be latched into one another by second latching elements 11.

The latching elements 10, 10a, 10b of the casing parts 9a, 9b and the latching elements 11, 11a, 11b of the locking element parts 6a, 6b are preferably designed as screw threads 31 that are formed in part in the circumferential direction or as teeth that are formed in part. The latching elements 10, 10a, 10b, 11a, 11b are arranged on the corresponding casing parts 9a, 9b and locking element parts 6a, 6b such that at least one casing part 9a and one of the locking element parts 6a can be adjusted together relative to the other casing and locking element parts 9b, 6b. By the coupling according to the invention of the length adjustment of casing parts 9a and locking element parts 6a, when adjusting the length of the locking unit 2, locking is also made possible by means of the locking elements 6, since the locking element 6 changes its length analogously to the length adjustment of the casing 9.

FIG. 7 is a view of a detail of the casing part 9a together with the latching elements 10 in the form of screw threads or teeth 31 that are formed in part. The screw threads 31 are only formed on two opposite sides of the casing part 9a. In the corresponding casing part 9b according to FIG. 6 that is also shown, the screw threads 31 are accordingly likewise only formed on two opposite sides of the casing part 9b. In one casing part 9a, the screw threads 31 form an outer thread, and in the other casing part 9b, the screw threads 31 form an inner thread or inner teeth. In the closed position, the screw threads 31 or teeth of the two casing parts 9a, 9b engage in one another and thus do not allow any length adjustment. For the length adjustment, the position of the casing parts 9a, 9b is rotated by 90°, such that the screw threads precisely no longer engage in one another and length adjustment is possible.

FIG. 8 is a view of a detail of the locking element part 6a together with the latching elements 11a in the form of screw threads or teeth 31 that are formed in part in the circumferential direction as an outer thread/outer teeth.

FIG. 9 is a view of a detail of the locking element part 6b together with the latching elements 11b in the form of screw threads 31 that are formed in part in the circumferential direction as an inner thread/inner teeth.

The interaction between the inner and outer threads/teeth 31 of the locking element parts 6a, 6b functions similarly to the interaction of the casing parts 9a, 9b. In a latching position, the screw threads 31 of the two locking element parts 6a, 6b engage in one another and thus do not allow any length adjustment. For the length adjustment, the position of the locking element parts 6a, 6b is rotated by 90°, such that the screw threads 31 precisely no longer engage in one another and length adjustment is possible. The rotational movements of the locking element parts 6a, 6b and the casing parts 9a, 9b are coupled via a connection of the casing part 9a and the locking element parts 6a, such that a length adjustment of the locking unit 2 always causes a length adjustment of the casing portion 30b and the corresponding locking element 6a, 6b.

FIG. 10 is a view of a preferred embodiment of the instrument holder 17 according to the invention, comprising a housing 18 and preferably three sliders 20. In addition, a holder is provided below the housing 18, by means of which the instrument holder 17 can be received in the hole 13 (FIG. 3) in the tool mount 12 (FIG. 3), which has already been described.

FIG. 11 is a partial section through the housing 18 of the instrument holder 17. The housing 18 comprises first openings 21, which are preferably designed as T-shaped guide rails 21 and extend in the manner of spokes in a straight line from the central cut-out 30 to the edge of the housing 18. The guide rails 21 are preferably arranged at an angle of 120° relative to one another. A substantially cuboid slider 20 runs in each of the T-shaped guide rails 21. Each slider comprises two lateral protrusions 32, which are received by the T-shaped guide rails 21 of the housing 18.

FIG. 12 shows the flat thread 19, which is arranged so as to be rotatable in the housing 18 of the instrument holder 17. The flat thread 19 has rotary means 22 that make it possible to rotate the flat thread 19. The rotary means 22 are preferably accessible from outside the housing 18 via at least one second opening 23 in the housing 18 of the instrument holder 17.

The inner end of the slider 20, which is within the housing 18, extends into the flat thread 19 and engages with a protrusion therein. The outer end of the slider 20 extends out of the housing 18. The slider comprises gripping surfaces 33 towards the center of the instrument holder 17, which surfaces can be enlarged in order to provide a larger projecting surface for holding an instrument.

The sliders 20 can be moved towards one another by rotation of the flat thread 19 in a first rotational direction and can be moved away from one another by rotation of the flat thread 19 in a second rotational direction counter to the first rotational direction.

Cut-outs 31 may preferably be arranged centrally in the base and cover of the housing 18 and at a corresponding point in the flat thread 19 such that an instrument to be clamped, for example a needle, can extend through this cut-out in the instrument holder 17.

The holding device 1 according to the invention can be formed by a plurality of locking units 2, both according to the first embodiment and the second embodiment. For this purpose, the locking units 2 can be combined with one another in the form of an assembly to form the holding device 1. A connector element 4 comprising at least two joint heads 5 is arranged between each of the thus combined locking units 2, with one joint head 5 being received in a socket 3 of the combined locking units 2 in each case.

### LIST OF REFERENCE SIGNS

1 holding device
2 locking unit
3 sockets
4 connector element
5 joint head
6 locking element (6a, 6b=locking element parts)
7 eccentric element
8 locking lever
9 casing (9a, 9b=casing parts)
10 first latching element
11 second latching element
12 tool mount
13 hole
14 locking shaft
15 unlocking element
16 spring
17 instrument holder
18 housing
19 flat thread
20 slider
21 first openings
22 rotary means
23 second openings
24 fixing unit
25 guide
26 screw
27 projection
28 notch
29 pivot axis
30 casing portions (a, b)
31 cut-out (FIG. 11) and thread (FIGS. 6, 7, 8 and 9)
32 lateral protrusions
33 gripping surface

1. Holding device (1) comprising at least one locking unit (2) having at least two sockets (3), wherein the sockets (3) are designed to make it possible for joint heads (5) received in the sockets (3) to each pivot about at least one pivot axis.

2. Holding device (1) according to claim 1, wherein the sockets (3) are designed to be rotatable relative to one another such that the orientation of the pivot axes of the sockets (3) relative to one another can be changed.

3. Holding device (1) according to claim 1, comprising at least one connector element (4) having at least two joint heads (5), wherein at least one joint head (5) is received in a socket (3).

4. Holding device (1) according to claim 3, comprising at least two locking units (2) and at least one connector element (4), wherein the joint heads (5) of the connector element (4) are each received in a socket (3) of a locking unit (2).

5. Locking unit (2) comprising at least two sockets (3), wherein the sockets (3) make it possible for joint heads (5) received in the sockets (3) to each pivot about at least one pivot axis, and comprising at least two locking elements (6), wherein the locking elements (6) lock joint heads (5) received in the sockets (3) in a locking position and allow joint heads (5) received in the sockets (3) to move freely about the pivot axes in an open position.

6. Locking unit (2) according to claim 5, wherein the sockets (3) are designed to be rotatable relative to one another in the open position such that the orientation of the pivot axes of the sockets (3) relative to one another can be changed, wherein the locking elements (6) lock the sockets (3) to one another in the locking position such that the position of the pivot axes can be fixed in the locking position.

7. Locking unit (2) according to claim 5, wherein, in the locking position, the locking elements (6) are pressed against joint heads (5) received in the sockets (3) by eccentrically arranged eccentric elements (7) of a locking lever (8).

8. Locking unit (2) according to claim 5, wherein the locking unit (2) is adjustable in length between the sockets (3).

9. Locking unit (2) according to claim 8, comprising a casing (9) made up of at least two casing parts (9a, 9b), wherein the at least two casing parts (9a, 9b) are designed to be adjustable relative to one another for the length adjustment of the locking unit (2).

10. Locking unit (2) according to claim 9, wherein the at least two casing parts (9a, 9b) can be latched into one another by latching elements (10a, 10b).

11. Locking unit (2) according to claim 8, wherein at least one of the locking elements (6) comprises a first and a second locking element part (6a, 6b), wherein the first and the second locking element part (6a, 6b) are designed to be adjustable relative to one another for the length adjustment of the locking unit (2).

12. Locking unit (2) according to claim 11, wherein the first and the second locking element part (6a, 6b) can be latched into one another by latching elements (11a, 11b).

13. Locking unit (2) according to claim 12, wherein the latching elements (10, 11) are designed as form-fitting elements (31) that are formed in part in the circumferential direction.

14. Locking unit (2) according to claim 8, wherein at least one casing part (9a) and one of the locking element parts (6a) can be adjusted together relative to the other casing and locking element parts (9b, 6b).

15. Holding device (1) according to claim 1, comprising a locking unit (2).

16. Tool mount (12) comprising at least one hole (13) for receiving a tool, wherein, in a closed position, at least one locking shaft (14) engages in the hole (13) and the tool can thus be fixed in the hole (13), wherein, in an open position, the locking shaft (14) does not engage in the hole (13) and the tool can thus be removed from the hole (13).

17. Tool mount (12) according to claim 16, wherein the locking shaft (14) can be moved from a closed position into an open position by an unlocking mechanism that can be released by an unlocking element (15).

18. Tool mount (12) according to claim 17, wherein the unlocking mechanism comprises a spring (16), which moves the locking shaft (14) into the closed position by means of spring force.

19. Holding device (1) according to claim 1, comprising a tool mount (12).

20. Instrument holder (17) comprising a housing (18), wherein at least one rotatable flat thread (19) is arranged in the housing (18), wherein a plurality of sliders (20) are arranged on the flat thread (19) and penetrate through the housing (18) through first openings (21) in the housing (18), wherein the sliders (20) can be moved towards one another by rotation of the flat thread (19) in a first rotational direction and can be moved away from one another by rotation of the flat thread (19) in a second rotational direction counter to the first rotational direction.

21. Instrument holder (17) according to claim 20, wherein the flat thread (19) has rotary means (22) that make it possible to rotate the flat thread (19), wherein the rotary means (22) are accessible from the outside via at least one opening (23) in the housing (18).

22. Holding device (1) according to claim 1, comprising an instrument holder (17).
